(19) United States
(12) Patent Application Publication
(10) Pub. No.: US 2011/0003324 A1
(43) Pub. Date: Jan. 6, 2011

(54) MICROFLUIDIC DEVICE HAVING ONBOARD TISSUE OR CELL SAMPLE HANDLING CAPABILITY

(76) Inventor: Gary P. Durack, Urbana, IL (US)

Correspondence Address: Woodard, Emhardt, Moriarty, McNett & Henry LLP;Sony Corporation, 111 Monument Circle, Suite 3700, Indianapolis, IN, 46204-5137, US

(21) Appl. No.: 12/830,887

(22) Filed: Jul. 6, 2010

Related U.S. Application Data
(60) Provisional application No. 61/223,082, filed on Jul. 6, 2009.
(60) Provisional application No. 61/223,083, filed on Jul. 6, 2009.
(60) Provisional application No. 61/223,093, filed on Jul. 6, 2009.

(51) Int. Cl. C12Q 1/02; C12M 1/00

(52) U.S. Cl. 435/29; 435/287.1

(57) ABSTRACT

The present disclosure is generally directed to systems for the storage and preservation of an original tissue or cell sample onboard a microfluidic device, such as a cytometry chip. In some embodiments, the sample may be disassociated while onboard the microfluidic device.

### CROSS-REFERENCE TO RELATED APPLICATIONS

The present application claims the benefit of the following: U.S. Provisional Patent Application No. 61/223,082, which was filed Jul. 6, 2009, U.S. Provisional Application No. 61/223,083, filed Jul. 6, 2009, and U.S. Provisional Application No. 61/223,093, filed Jul. 6, 2009. All of these applications are incorporated herein by reference in their entireties

### TECHNICAL FIELD OF THE DISCLOSURE

The present disclosure relates generally to microfluidic cytometry systems and, more particularly, to a microfluidic device having onboard tissue or cell sample handling capability.

### BACKGROUND OF THE DISCLOSURE

Flow cytometry-based cell sorting was first introduced to the research community more than 20 years ago. It is a technology that has been widely applied in many areas of life science research, serving as a critical tool for those working in fields such as genetics, immunology, molecular biology and environmental science. Unlike bulk cell separation techniques such as immuno-panning or magnetic column separation, flow cytometry-based cell sorting instruments measure, classify and then sort individual cells or particles serially at rates of several thousand cells per second or higher. This rapid “one-by-one” processing of single cells has made flow cytometry a unique and valuable tool for extracting highly pure sub-populations of cells from otherwise heterogeneous cell suspensions.

Cells targeted for sorting are usually labeled in some manner with a fluorescent material. The fluorescent probes bound to a cell emit fluorescent light as the cell passes through a tightly focused, high intensity, light beam (typically a laser beam). A computer records emission intensities for each cell. These data are then used to classify each cell for specific sorting operations. Flow cytometry-based cell sorting has been successfully applied to hundreds of cell types, cell constituents and microorganisms, as well as many types of inorganic particles of comparable size.

Flow cytometers are also applied widely for rapidly analyzing heterogeneous cell suspensions to identify constituent sub-populations. Examples of the many applications where flow cytometry cell sorting is finding use include isolation of rare populations of immune system cells for AIDS research, isolation of genetically atypical cells for cancer research, isolation of specific chromosomes for genetic studies, and isolation of various species of microorganisms for environmental studies. For example, fluorescently labeled monoclonal antibodies are often used as “markers” to identify immune cells such as T lymphocytes and B lymphocytes, clinical laboratories routinely use this technology to count the number of “CD4 positive” T cells in HIV infected patients, and they also use this technology to identify cells associated with a variety of leukemia and lymphoma cancers.

Recently, two areas of interest are moving cell sorting towards clinical, patient care applications, rather than strictly research applications. First is the move away from chemical pharmaceutical development to the development of biopharmaceuticals. For example, the majority of novel cancer therapies are now biologics containing proteins or peptides. These include a class of antibody-based cancer therapeutics. Cytometry-based cell sorters can play a vital role in the identification, development, purification and, ultimately, production of these products.

There is also a move toward the use of cell replacement therapy for patient care. Much of the current interest in stem cells revolves around a new area of medicine often referred to as regenerative therapy or regenerative medicine. These therapies may often require that large numbers of relatively rare cells be isolated from sample patient tissue. For example, adult stem cells may be isolated from bone marrow or adipose tissue and ultimately used as part of a re-infusion back into the patient from whom they were removed. Cytometry lends itself very well to such therapies.

There are two basic types of cell sorters in wide use today. They are the “droplet cell sorter” and the “fluid switching cell sorter.” The droplet cell sorter utilizes micro-droplets as containers to transport selected cells to a collection vessel. The micro-droplets are formed by coupling ultrasonic energy to a jetting stream. Droplets containing cells selected for sorting are then electrostatically steered to the desired location. This is a very efficient process, allowing as many as 90,000 cells per second to be sorted from a single stream, limited primarily by the frequency of droplet generation and the time required for illumination.

A detailed description of a prior art flow cytometry system is given in United States Published Patent Application No. US 2005/0112541 A1 to Durack et al.

Droplet cell sorters, however, are not particularly biosafe. Aerosols generated as part of the droplet formation process can carry biohazardous materials. Because of this, biosafe droplet cell sorters have been developed that are contained within a biosafety cabinet so that they may operate within an essentially closed environment. Unfortunately, this type of system does not lend itself to the sterility and operator protection required for routine sorting of patient samples in a clinical environment.

The second type of flow cytometry-based cell sorter is the fluid switching cell sorter. Most fluid switching cell sorters utilize a piezoelectric device to drive a mechanical system which diverts a segment of the flowing sample stream into a collection vessel. Compared to droplet cell sorters, fluid switching cell sorters have a lower maximum cell sorting rate due to the cycle time of the mechanical system used to divert the sample stream. This cycle time, the time between initial sample diversion and when stable non-sorted flow is restored, is typically significantly greater than the period of a droplet generator on a droplet cell sorter. This longer cycle time limits fluid switching cell sorters to processing rates of several hundred cells per second. For the same reason, the stream segment switched by a fluid cell sorter is usually at least ten times the volume of a single micro-drop from a droplet generator. This results in a correspondingly lower concentration of cells in the fluid switching sorter's collection vessel as compared to a droplet sorter's collection vessel.

Newer generation microfluidics technologies offer great promise for improving the efficiency of fluid switching devices and providing cell sorting capability on a chip similar in concept to an electronic integrated circuit. Many microfluidic systems have been demonstrated that can successfully sort cells from heterogeneous cell populations. They have the advantages of being completely self-contained, easy to sterilize, and can be manufactured on sufficient scales (with the resulting manufacturing efficiencies) to be considered a disposable part.

A generic microfluidic device is illustrated in FIG. 1 and indicated generally at 10. The microfluidic device 10 comprises a substrate 12 having a fluid flow channel 14 formed therein by any convenient process as is known in the art. The substrate 12 may be formed from glass, plastic or any other convenient material, and may be substantially transparent or substantially transparent in a portion thereof. In certain embodiments, the substrate 12 is injection molded. In certain embodiments, the substrate 12 comprises industrial plastic such as a Cyclo Olefin Polymer (COP) material, or other plastic. As a result, the substrate 12 is transparent such that a cytometry optics module can analyze the sample fluid stream as described further below. In one embodiment, the microfluidic device 10 is disposable.

The substrate 12 further has three ports 16, 18 and 20 coupled thereto. Port 16 is an inlet port for a sheath fluid. Port 16 has a central axial passage that is in fluid communication with a fluid flow channel 22 that joins fluid flow channel 14 such that sheath fluid entering port 16 from an external supply (not shown) will enter fluid flow channel 22 and then flow into fluid flow channel 14. The sheath fluid supply may be attached to the port 16 by any convenient coupling mechanism as is known to those skilled in the art. In one embodiment, the sheath fluid comprises a buffer or buffered solution. For example, the sheath fluid comprises 0.96% Dulbecco's phosphate buffered saline (w/v), 0.1% BSA (w/v), in water at a pH of about 7.0.

Port 18 also has a central axial passage that is in fluid communication with a fluid flow channel 14 through a sample injection tube 24. Sample injection tube 24 is positioned to be coaxial with the longitudinal axis of the fluid flow channel 14. Injection of a liquid sample of cells into port 18 while sheath fluid is being injected into port 16 will therefore result in the cells flowing through fluid flow channel 14 surrounded by the sheath fluid. The dimensions and configuration of the fluid flow channels 14 and 22, as well as the sample injection tube 24 are chosen so that the sheath/sample fluid will exhibit laminar flow as it travels through the device 10, as is known in the art. Port 20 is coupled to the terminal end of the fluid flow channel 14 so that the sheath/sample fluid may be removed from the microfluidic device 10.

While the sheath/sample fluid is flowing through the fluid flow channel 14, it may be analyzed using cytometry techniques by shining an illumination source through the substrate 12 and into the fluid flow channel 14 at some point between the sample injection tube 24 and the outlet port 20. Additionally, the microfluidic device 10 could be modified to provide for a cell sorting operation, as is known in the art.

Although basic microfluidic devices similar to that described hereinabove have been demonstrated to work well, there is a need in the prior art for improvements to cytometry systems employing microfluidic devices. The present invention is directed to meeting this need.

### SUMMARY OF THE DISCLOSURE

The present disclosure is generally directed to systems for the storage and preservation of an original tissue or cell sample onboard a microfluidic device, such as a cytometry chip. In some embodiments, the sample may be disassociated while onboard the microfluidic device.

In one embodiments, a microfluidic device is disclosed, comprising a substrate, a microfluidic flow channel formed in said substrate, wherein said flow channel extends through a portion of said substrate adapted to facilitate cytometry analysis of cells flowing in said flow channel, and a sample repository onboard said substrate and containing material operative to preserve cells in a tissue sample placed within said sample repository.

In another embodiment, a method for analyzing cells is disclosed, comprising the steps of a) providing a tissue sample; b) disassociating cells from said tissue sample; c) analyzing said disassociated cells by cytometry while said cells are onboard a microfluidic device having a substrate; and d) placing a non-disassociated portion of said tissue sample in a sample repository onboard said microfluidic device.

In another embodiment, a microfluidic device is disclosed, comprising a substrate, a sample well onboard said substrate for holding a tissue sample, means for disassociating cells from said tissue sample while said tissue sample is in said sample well, and a microfluidic flow channel formed in said substrate and operatively coupled to said sample well for receiving said disassociated cells, wherein said flow channel extends through a portion of said substrate adapted to facilitate cytometry analysis of said cells flowing in said flow channel

In yet another embodiment, a method for analyzing cells is disclosed, comprising the steps of: a) placing a tissue sample in a sample well onboard a microfluidic device; b) disassociating cells from said tissue sample within said sample well; and c) analyzing said disassociated cells by cytometry while said cells are onboard said microfluidic device.

In still another embodiment, a microfluidic device is disclosed, comprising a substrate, an input port operatively coupled to said substrate for accepting a quantity of cells, a microfluidic flow channel formed in said substrate, wherein said flow channel extends through a portion of said substrate adapted to facilitate cytometry analysis of said cells flowing in said flow channel, and a sample repository onboard said substrate and in fluid communication with said microfluidic flow channel, wherein a portion of said cells may be routed to said sample repository through said flow channel without undergoing cytometry analysis.

In another embodiment, a method for analyzing cells is disclosed, comprising the steps of: a) providing a quantity of cells into a microfluidic flow channel formed in a substrate of a microfluidic device; b) depositing a first portion of said cells in a sample well onboard said substrate and in fluid communication with said microfluidic flow channel; and c) analyzing a second portion of said cells by cytometry while said cells are onboard a microfluidic device.

Other embodiments are also disclosed.

### BRIEF DESCRIPTION OF THE DRAWINGS

FIG. 1 is a perspective view of a prior art microfluidic device.

FIG. 2 is a schematic perspective view of a microfluidic device according to an embodiment of the present disclosure.

FIGS. 3A-D are schematic perspective views of exemplary means for forming a sample repository well on a microfluidic device.

FIG. 4 is a schematic perspective view of a microfluidic device according to an embodiment of the present disclosure.

FIG. 5 is a schematic perspective view of a microfluidic device according to an embodiment of the present disclosure.

FIG. 6 is a schematic perspective view of a microfluidic device according to an embodiment of the present disclosure.

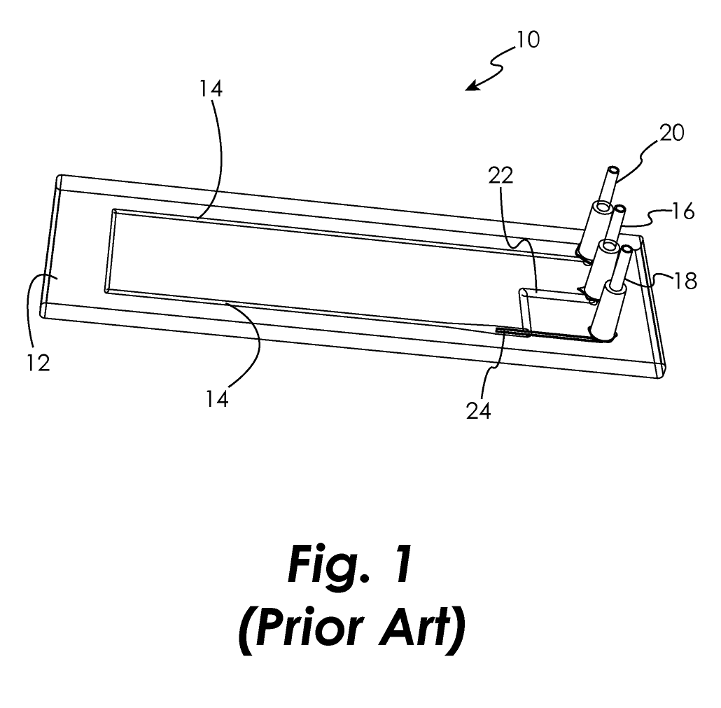

### DESCRIPTION OF THE ILLUSTRATED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the disclosure is thereby intended, such alterations and further modifications in the illustrated device, and such further applications of the principles of the disclosure as illustrated therein are contemplated as would normally occur to one skilled in the art to which the disclosure relates.

The present disclosure is generally directed to systems for the storage and preservation of an original tissue or cell sample on a microfluidic device, such as a cytometry chip. In some embodiments, the sample may be disassociated while on board the microfluidic device.

### Microfluidic Devices Having Tissue Sample Storage

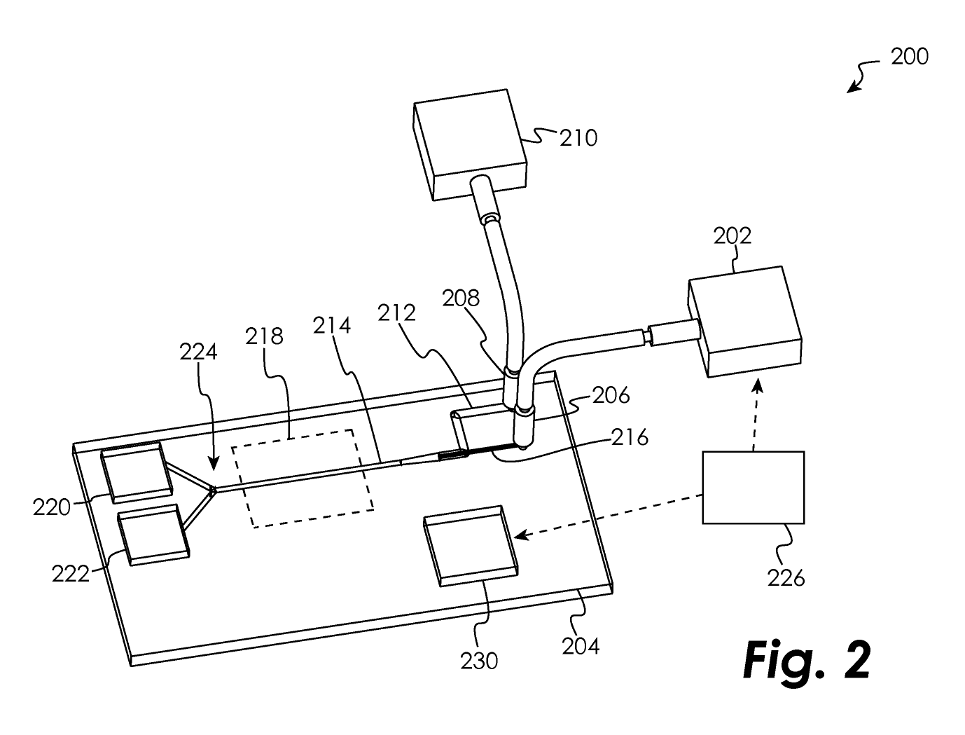

In a first embodiment, the microfluidic device has the capability of storing and preserving a tissue sample, for example a tissue sample taken from the same tissue where the cell supply for the cytometry process originates, onboard the microfluidic device. FIG. 2 schematically illustrates a system 200 in which cells coming from an external cell supply 202 are analyzed via cytometry using a microfluidic device formed onboard (i.e. on and/or in) substrate 204. As used herein, the term “onboard” is intended to encompass a structure that is carried by the substrate, whether that structure is on the substrate, in the substrate, or partially on and partially in the substrate. Cells from external supply 202 are input to the microfluidic device 200 through an input port 206. Port 208 is an inlet port for a sheath fluid from sheath fluid supply 210. Port 208 has a central axial passage that is in fluid communication with a fluid flow channel 212 such that sheath fluid entering port 208 from external supply 210 will enter fluid flow channel 212 and then flow into the main fluid flow channel 214. The sheath fluid supply 210 may be attached to the port 208 by any convenient coupling mechanism as is known to those skilled in the art. In other embodiments, a system that does not require sheath flow can be employed.

Port 206 also has a central axial passage that is in fluid communication with a fluid flow channel 214 through a sample injection tube 216. Sample injection tube 216 is positioned to be coaxial with the longitudinal axis of the fluid flow channel 214. Injection of a liquid sample of cells from cell supply 202 into port 206 while sheath fluid is being injected into port 208 will therefore result in the cells flowing through fluid flow channel 214 surrounded by the sheath fluid. The dimensions and configuration of the fluid flow channels 214 and 212, as well as the sample injection tube 216 are chosen so that the sheath/sample fluid will exhibit laminar flow as it travels through the device 200, as is known in the art.

Cytometry analysis, possibly using a device external to the microfluidic device, may be performed in analysis section 218 (the specific operations that occur in analysis section 218 are not critical to the present disclosure). As a result of the analysis performed in section 218, the cells may optionally be sorted into different sample wells 220 or 222 based on differing characteristics of the cells. Sorting of the cells may be achieved by proper control of valve 224, as is known in the art. In certain embodiments, the sample wells 220, 222 have outlet ports (not shown) in fluid communication therewith in order to facilitate removal of the sorted sample from the wells.

In certain embodiments, cells may be sorted into different sample wells based on the intended future use for the cells. For example, cells having the same characteristics, or phenotype, may be sorted into one well where they are fixed for viewing, and sorted into another well where they are maintained in a viable state to undergo additional functional measurements. In other embodiments, the cells may be deposited into the wells based upon volume as opposed to a sorting method. For simplicity and ease of illustration, FIG. 2 schematically shows single channels extending between the components, areas or sections of device 200. However, it should be appreciated that the single channels may be representative of multiple cytometry channels and a variety of possible configurations of channels as would occur to one skilled in the art.

In certain situations, it may be desirable to retain a sample of tissue from which the cells in cell supply 202 were obtained. In order to facilitate this, a tissue sample taken from original tissue 226 may be placed in a sample repository 230 located onboard (i.e. on and/or in) the substrate 204, to be stored and optionally preserved, using chemicals or other means, for later viewing, imaging or testing by a researcher or medical professional. Accordingly, the cells contained in the tissue sample placed in repository 230 are not initially analyzed via cytometry at analysis section 218. As illustrated, the cells from cell supply 202 which are analyzed via the cytometry process and the tissue sample placed in repository 230 may both be taken from the same original tissue 226. This provides a researcher or medical professional with the ability to view the cells as they naturally occur within the tissue by viewing the tissue sample in repository 230, rather than viewing the individual cells after they have been manipulated to disassociate them from the tissue 226 and have gone through the cytometry process. In other words, the researcher or medical professional is provided with the ability to perform both analysis via the cytometry analysis and observation or morphological review of cells which originate from the same tissue. In one particular example, the tissue sample take from tissue 208 and placed in repository 230 may be a thin section of tissue taken from a biopsy suspected of containing a cancerous malignancy.

If the results of the cytometry process indicate a problem or potential problem with the cells from the cell supply 202, the researcher or medical professional can view the cells which originate from the same tissue specimen 226 as the cells that were analyzed via cytometry, by viewing the sample of original tissue in repository 230. Viewing can be done with either a traditional optical microscope or with an electronic image analysis system. Additionally, the researcher or medical professional can perform additional testing on cells originating from the same tissue, if necessary, by disassociating the cells from the tissue sample in repository 230 and operating the cytometry process or other appropriate test. Furthermore, the archived tissue sample in sample repository 230 may be subject to other tests that do not require cell disassociation. In this manner, rapid screening of the sample can be accomplished using the flow cytometry analysis and sorting. Subsequently, those samples identified as suspect by the flow cytometry screeing can be examined in detail using image cytometry techniques. The microfluidic device provides a convenient and useful method to contain, store, and transport all cells collected from the patient sample. Such a device could easily be archived for permanent storage if desired.

The repository 230 is shown as being positioned near the top of the device 200; however, it should be appreciated that the repository may be positioned elsewhere on and/or in the substrate 204. In some embodiments, the repository 230 may contain the necessary reagents and/or chemicals therein to fix the cells in the tissue sample in their current state for an extended period of time to maintain the morphology and integrity of the tissue sample for later observation or testing by a researcher or medical professional. In some embodiments, these reagents and/or chemicals are placed within the repository 230 when the device 200 is manufactured. In other embodiments, the reagents and/or chemicals may be placed within the repository 230 prior to or just after placement of a tissue sample within the repository 230.

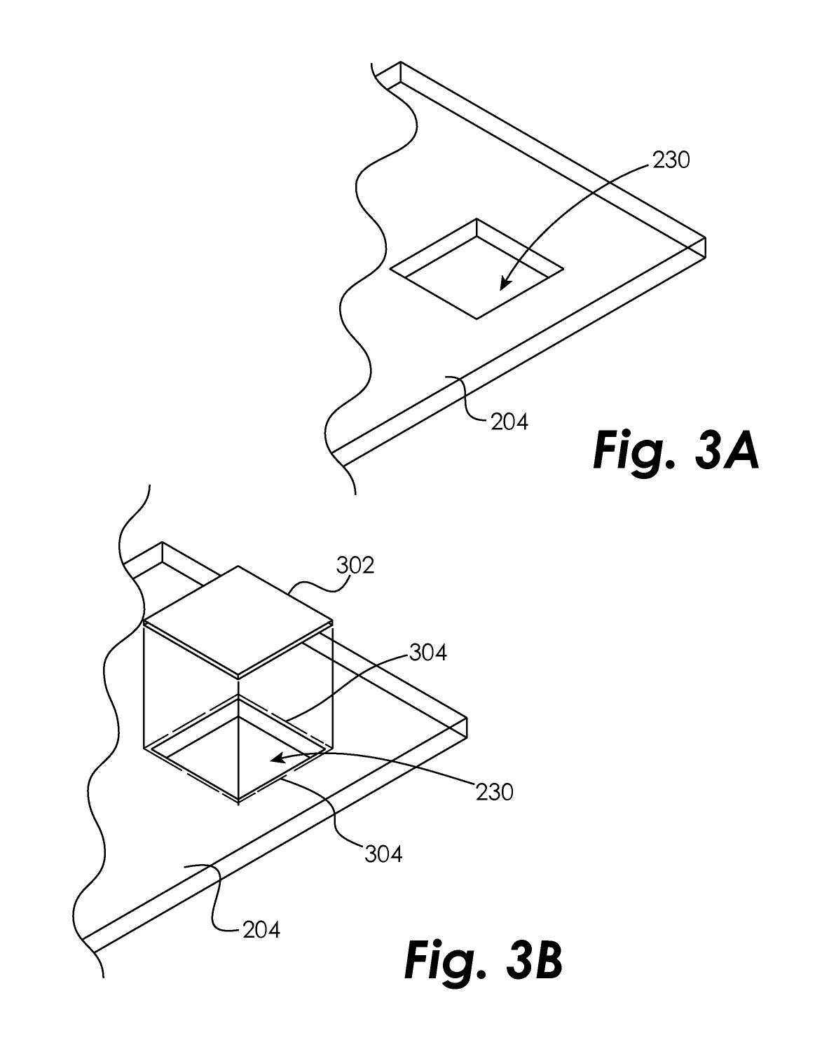

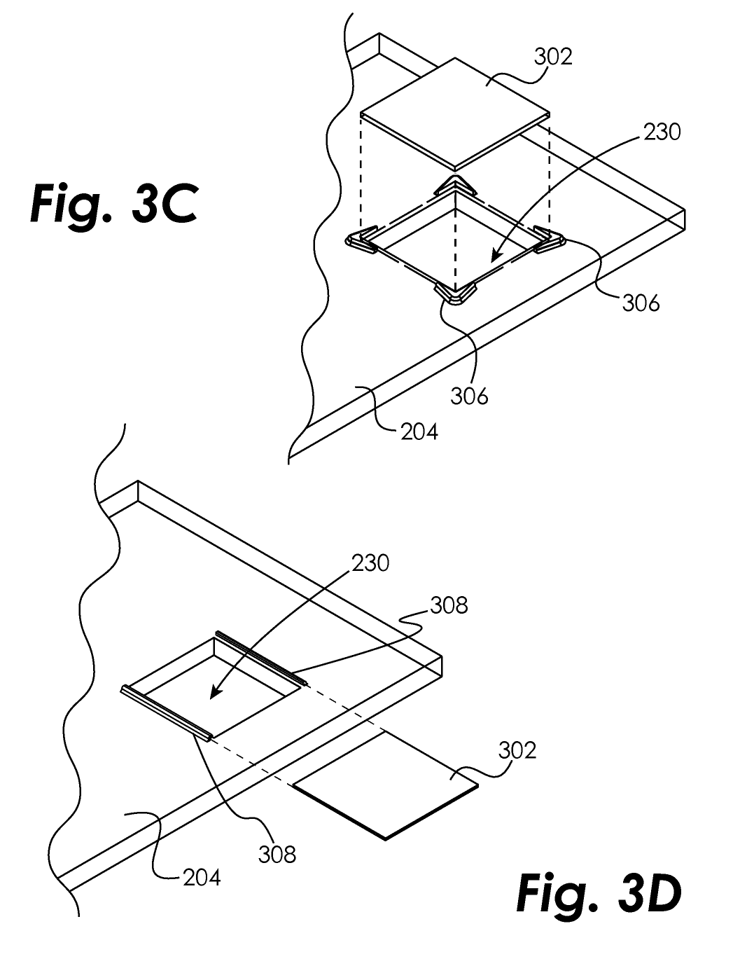

As shown in FIGS. 3A-D, the sample repository 230 may take any convenient physical form, such as an open well 230 formed into the surface of the substrate 204, which may remain open as shown in FIG. 3A. In certain embodiments, the sample repository 230 may include a cover 302 that is glued in place by means of an adhesive 304 placed on the surface of the substrate 204. In certain embodiments, the adhesive 304 is placed upon the surface of the substrate 204 when it is manufactured and is covered by a release layer that may be removed prior to adhering the cover 302 to the substrate 204, as illustrated in FIG. 3B. In other embodiments, the cover 302 may be snapped in place with resilient members 306 that engage the substrate 204 and provide an interference fit when the cover 302 is snapped into place, as illustrated in FIG. 3C. In other embodiments, the cover 302 may be slid into place under guides 308 that extend from the substrate 204 surface, as illustrated in FIG. 3D. The examples of FIGS. 3A-D are given by way of non-limiting example only, and the present disclosure comprehends any other convenient means as would occur to one of ordinary skill in the art. The above examples are intended to be only non-limiting examples of many possible configurations.

### Microfluidic Devices Having Tissue Disassociation Means

Certain other embodiments of the present disclosure are generally directed to microfluidic devices, such as cytometry chips, which allow for disassociation of cell suspensions from tissue samples and analysis of the disassociated cells via cytometry, such as flow or image cytometry as non-limiting examples. The cells may be disassociated from the tissue sample by using chemical, mechanical and/or vibratory techniques.

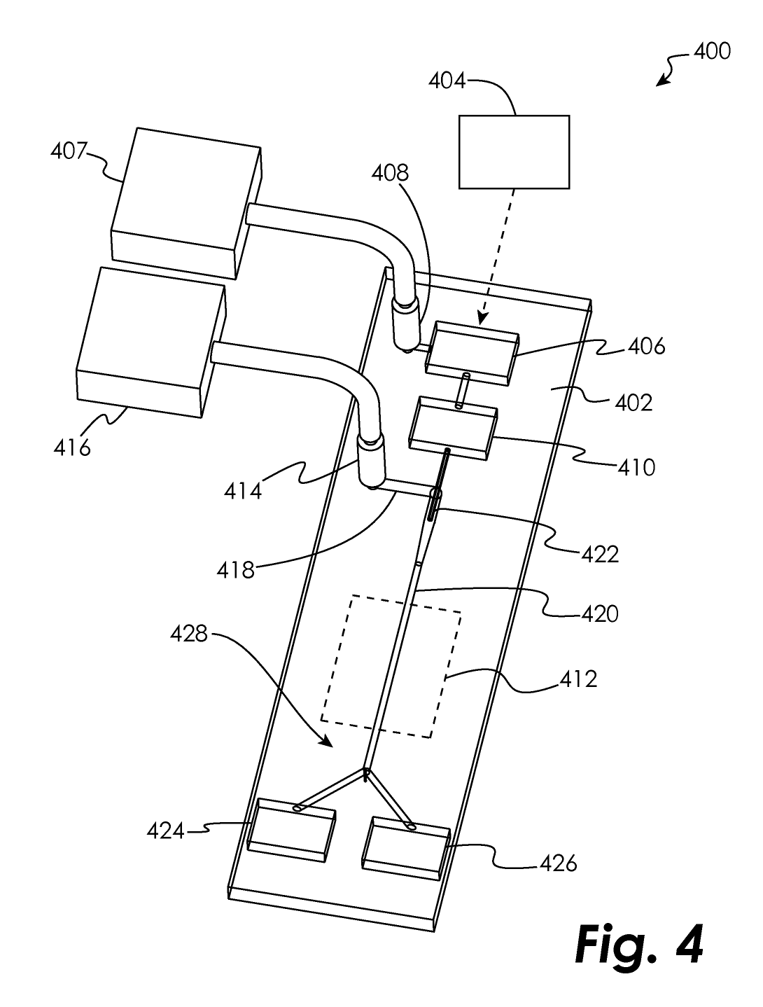

FIG. 4 schematically illustrates a system 400 where chemical techniques are applied to a tissue sample to disassociate a cell sample for the cytometry process. A tissue sample is taken from original tissue 404 and placed into tissue sample well 406 on microfluidic device 402. Chemicals 407 may then be applied to the tissue sample in the well 406 to at least partially digest the material holding the cells together in the tissue. In certain embodiments, the chemicals 407 can include the application of detergents and enzymes operable to break down the material, such as fibers, holding the cells together in the tissue sample, as is well known in the art. In certain embodiments, the chemicals are applied to the microfluidic device 402 from an external reservoir via a port 408 in fluid communication with tissue sample well 406 on the microfluidic device 402. In other embodiments, the chemicals 407 may be delivered to the tissue sample well 406 prior to placement of the tissue sample therein and the tissue sample may then be placed in the well. In such embodiments, the microfluidic device 402 may be packaged and sold with the chemicals in the tissue sample well 406 in a dried format. In certain embodiments the microfluidic device 402 can be inserted into an external machine which applies the chemicals 407 to disassociate the cells for the cytometry analysis, such as the introduction of chemicals by the machine into the tissue sample well 406 through the port 408. The machine may also assist in conducting the cytometry analysis with respect to the cell sample on the microfluidic device 402.

The chemicals 407 function to pull or disassociate cell sample 410 from tissue sample 406 for introduction into and analysis in the cytometry analysis section 412 (the specific operations that occur in analysis section 212 are not critical to the present disclosure). Port 414 is an inlet port for a sheath fluid from sheath fluid supply 416. Port 414 has a central axial passage that is in fluid communication with a fluid flow channel 418 such that sheath fluid entering port 414 from external supply 416 will enter fluid flow channel 418 and then flow into the main fluid flow channel 420. The sheath fluid supply 416 may be attached to the port 414 by any convenient coupling mechanism as is known to those skilled in the art.

Cell sample 410 also is in fluid communication with a fluid flow channel 420 through a sample injection tube 422. Sample injection tube 422 is positioned to be coaxial with the longitudinal axis of the fluid flow channel 420. Injection of a liquid sample of cells from cell sample 410 into sample injection tube 422 while sheath fluid is being injected into port 414 will therefore result in the cells flowing through fluid flow channel 420 surrounded by the sheath fluid. The dimensions and configuration of the fluid flow channels 418 and 420, as well as the sample injection tube 422 are chosen so that the sheath/sample fluid will exhibit laminar flow as it travels through the device 400, as is known in the art.

Cytometry analysis may be performed in analysis section 412. As a result of the analysis performed in section 412, the cells may optionally be sorted into different sample wells 424 or 426 based on differing characteristics of the cells. Sorting of the cells may be achieved by proper control of valve 428, as is known in the art. In certain embodiments, the sample wells 424, 426 have outlet ports (not shown) in fluid communication therewith in order to facilitate removal of the sorted sample from the wells.

In certain embodiments, cells may be sorted into different sample wells based on the intended future use for the cells. For example, cells having the same characteristics, or phenotype, may be sorted into one well where they are fixed for viewing, and sorted into another well where they are maintained in a viable state to undergo additional functional measurements. In other embodiments, the cells may be deposited into the wells based upon volume as opposed to a sorting method. For simplicity and ease of illustration, FIG. 4 schematically shows single channels extending between the components, areas or sections of device 400. However, it should be appreciated that the single channels may be representative of multiple cytometry channels and a variety of possible configurations of channels as would occur to one skilled in the art.

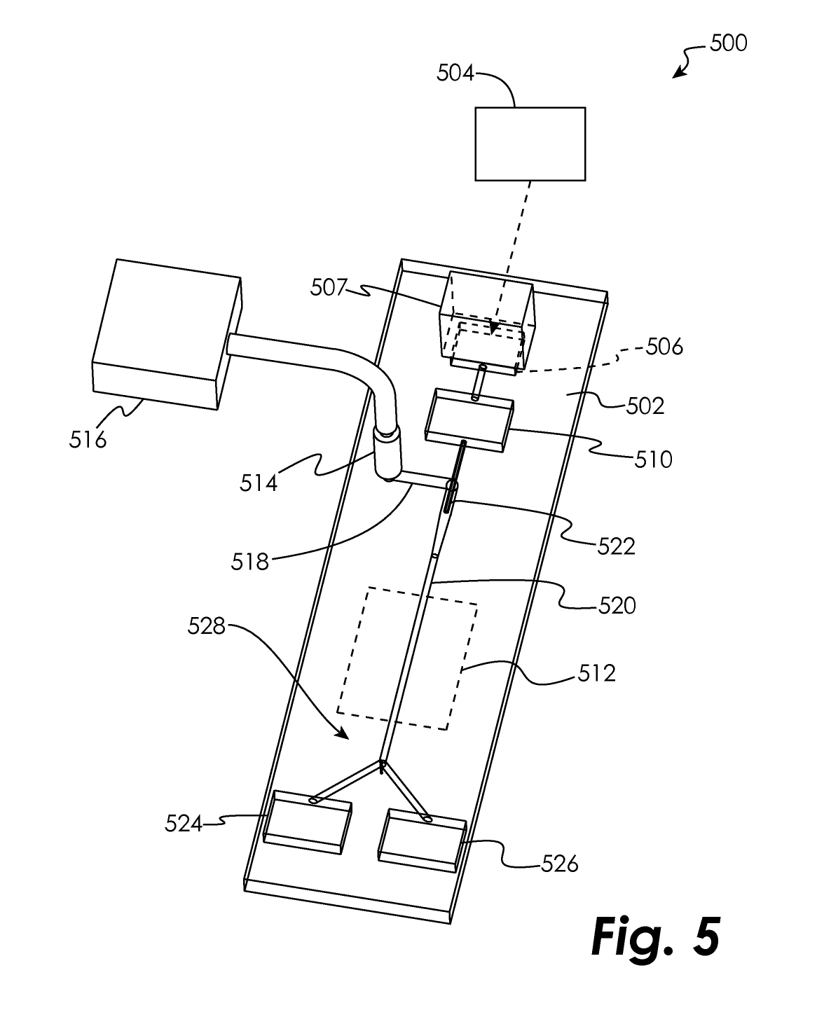

FIG. 5 schematically illustrates a system 500 where vibratory techniques, such as ultrasonic acoustic methods to name just one non-limiting example, are applied to a tissue sample to disassociate a cell sample for the cytometry process. A tissue sample is taken from original tissue 504 and placed into tissue sample well 506 on microfluidic device 502. A source of vibratory energy 507, such as a piezoelectric acoustic device to name just one non-limiting example, may be applied to the tissue sample in the well 506 to disassociate the cells from the tissue sample. Using the source of vibratory energy 507, a process of sonication may be applied to the tissue sample in well 506 where sound energy (such as, for example, ultrasonic energy) is applied in order to agitate the cells in the sample. It should be appreciated that the chemicals 407 and the vibratory energy 507 can both be used on the same microfluidic device, and can be applied substantially simultaneously or consecutively with either of the techniques applied first, in order to more effectively disassociate the cells from the tissue sample. Additionally, in certain embodiments the microfluidic device 502 can be inserted into an external machine which applies the techniques to disassociate the cells for the cytometry analysis, such as the introduction of chemicals by the machine into the tissue sample well 506 and/or the application of vibratory energy to the microfluidic device 502 by the machine. The machine may also assist in conducting the cytometry analysis with respect to the cell sample on the microfluidic device 502.

The vibratory technique 507 (sometimes in conjunction with the chemicals 407) functions to pull or disassociate cell sample 510 from tissue sample 506 for introduction into and analysis in the cytometry analysis section 512 (the specific operations that occur in analysis section 512 are not critical to the present disclosure). Port 514 is an inlet port for a sheath fluid from sheath fluid supply 516. Port 514 has a central axial passage that is in fluid communication with a fluid flow channel 518 such that sheath fluid entering port 514 from external supply 516 will enter fluid flow channel 518 and then flow into the main fluid flow channel 520. The sheath fluid supply 516 may be attached to the port 514 by any convenient coupling mechanism as is known to those skilled in the art.

Cell sample 510 also is in fluid communication with a fluid flow channel 520 through a sample injection tube 522. Sample injection tube 522 is positioned to be coaxial with the longitudinal axis of the fluid flow channel 520. Injection of a liquid sample of cells from cell sample 510 into sample injection tube 522 while sheath fluid is being injected into port 514 will therefore result in the cells flowing through fluid flow channel 520 surrounded by the sheath fluid. The dimensions and configuration of the fluid flow channels 518 and 520, as well as the sample injection tube 522 are chosen so that the sheath/sample fluid will exhibit laminar flow as it travels through the device 500, as is known in the art.

Cytometry analysis may be performed in analysis section 512. As a result of the analysis performed in section 512, the cells may optionally be sorted into different sample wells 524 or 526 based on differing characteristics of the cells. Sorting of the cells may be achieved by proper control of valve 528, as is known in the art. In certain embodiments, the sample wells 524, 526 have outlet ports (not shown) in fluid communication therewith in order to facilitate removal of the sorted sample from the wells.

In certain embodiments, cells may be sorted into different sample wells based on the intended future use for the cells. For example, cells having the same characteristics, or phenotype, may be sorted into one well where they are fixed for viewing, and sorted into another well where they are maintained in a viable state to undergo additional functional measurements. In other embodiments, the cells may be deposited into the wells based upon volume as opposed to a sorting method. For simplicity and ease of illustration, FIG. 5 schematically shows single channels extending between the components, areas or sections of device 500. However, it should be appreciated that the single channels may be representative of multiple cytometry channels and a variety of possible configurations of channels as would occur to one skilled in the art.

In other embodiments, a mechanical disassociation technique may applied to disassociate the cell sample from tissue sample, either in addition to or in lieu of one or both of the chemical and vibratory techniques. As an example, the mechanical technique can include the use of a micro electro-mechanical system operating a mechanical “flapper” member within the tissue sample well to physically break the tissue apart and disassociate the cells. However, it should be appreciated that the mechanical disassociation technique may include other appropriate mechanical devices operable to at least partially disassociate the cells from the tissue supply.

### Microfluidic Devices Having Cell Sample Storage

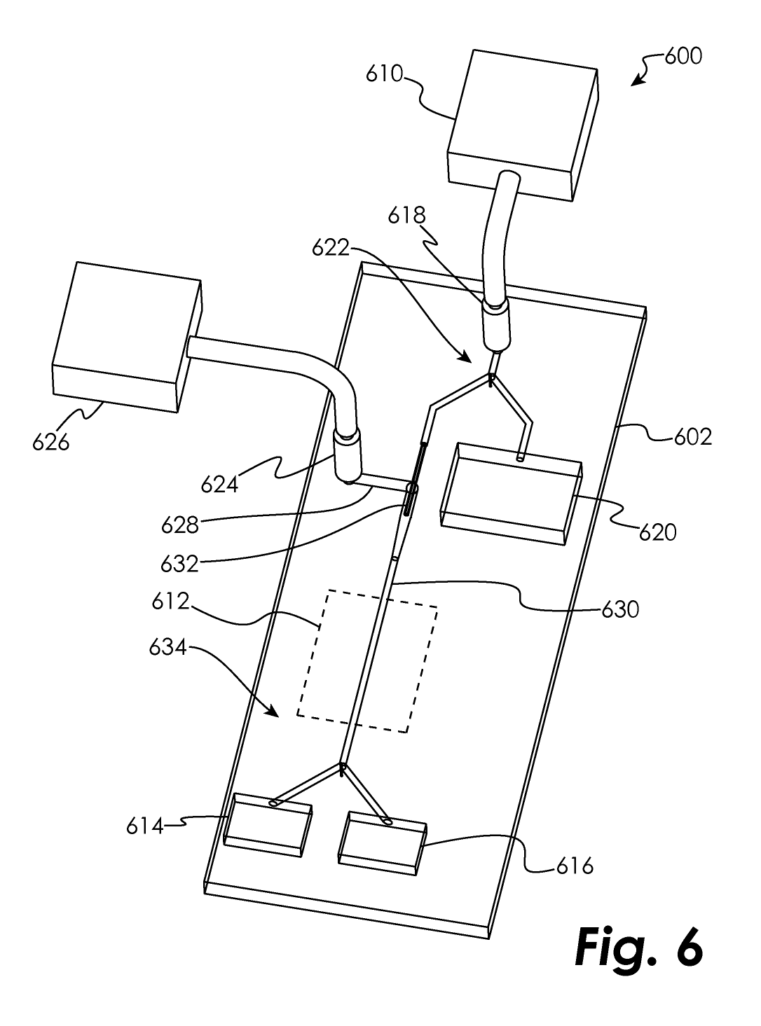

Certain embodiments of the present disclosure are generally directed to systems for the storage and preservation of an unaltered cell sample on a microfluidic device, such as a cytometry chip, the cell sample being a portion of the original cell supply taken before the cells undergo the cytometry analysis. In certain embodiments, the cytometry analysis is a flow cytometry analysis or image cytometry analysis. FIG. 6 schematically illustrates a system 600 in which cells from a cell supply 610 are analyzed via cytometry in analysis section 612 (the specific operations that occur in analysis section 612 are not critical to the present disclosure). According to the results of the analysis performed, the cells may be sorted into different chambers 614, 616.

Additionally, a sample from the original cell supply 610 may be diverted to a cell sample repository 620 prior to entry into analysis section 612 and preserved for later viewing, imaging or testing by a researcher or medical professional. Accordingly, the sample cells contained in the repository 620 are not initially analyzed via cytometry at analysis section 612. Cells from cell supply 610 are applied to input port 618 and a portion of the sample may be diverted into repository 620 via means for physically diverting the sample, such as the valve 622, as is known in the art. In certain embodiments, information obtained during the analysis section 612 may dictate the attention that is directed to the unaltered cell sample in repository 620. In other embodiments, information obtained during analysis in section 612 may dictate whether a separate cell sample is saved in the cell sample repository 620 at all. As an example, the cell sample in repository 620 may be frozen to preserve the sample for later use by a researcher or medical professional. Additionally, the cell sample may be otherwise stored for later attention, and the cell sample repository may have appropriate chemicals and/or reagents therein in order to help preserve the cell sample. In certain embodiments, the repository 620 may be detached from microfluidic device 602 and stored independently thereof or the whole microfluidic device 602 may be stored and/or transported as desired.

Port 624 is an inlet port for a sheath fluid from sheath fluid supply 626. Port 624 has a central axial passage that is in fluid communication with a fluid flow channel 628 such that sheath fluid entering port 624 from external supply 626 will enter fluid flow channel 628 and then flow into the main fluid flow channel 630. The sheath fluid supply 626 may be attached to the port 624 by any convenient coupling mechanism as is known to those skilled in the art.

Cell sample 610 that is destined for analysis section 612 is also in fluid communication with a fluid flow channel 630 when valve 622 is placed in the appropriate position. Cell sample 610 enters fluid flow channel 630 through a sample injection tube 632. Sample injection tube 632 is positioned to be coaxial with the longitudinal axis of the fluid flow channel 630. Injection of a liquid sample of cells from cell sample 610 into sample injection tube 632 while sheath fluid is being injected into port 624 will therefore result in the cells flowing through fluid flow channel 630 surrounded by the sheath fluid. The dimensions and configuration of the fluid flow channels 628 and 630, as well as the sample injection tube 632 are chosen so that the sheath/sample fluid will exhibit laminar flow as it travels through the device 600, as is known in the art.

Cytometry analysis may be performed in analysis section 612. As a result of the analysis performed in section 612, the cells may optionally be sorted into different sample wells 614 or 616 based on differing characteristics of the cells. Sorting of the cells may be achieved by proper control of valve 634, as is known in the art. In certain embodiments, the sample wells 614, 616 have outlet ports (not shown) in fluid communication therewith in order to facilitate removal of the sorted sample from the wells.

In certain embodiments, cells may be sorted into different sample wells based on the intended future use for the cells. For example, cells having the same characteristics, or phenotype, may be sorted into one well where they are fixed for viewing, and sorted into another well where they are maintained in a viable state to undergo additional functional measurements. In other embodiments, the cells may be deposited into the wells based upon volume as opposed to a sorting method. For simplicity and ease of illustration, FIG. 6 schematically shows single channels extending between the components, areas or sections of device 600. However, it should be appreciated that the single channels may be representative of multiple cytometry channels and a variety of possible configurations of channels as would occur to one skilled in the art.

The cell sample to be stored in repository 620 on microfluidic device 602 may be diverted from the channel, tube or pathway leading from the original cell supply 610 to analysis section 612, as schematically illustrated in FIG. 6. In other embodiments, the cell sample may be taken from the original cell supply 610 independent of the flow to the analysis section 612. The repository 620 is shown as being positioned near the middle of the microfluidic device 602; however, it should be appreciated that the repository may be positioned elsewhere on the device. In some embodiments, the repository 620 may contain the necessary reagents and/or other chemicals therein to fix the cells in the cell sample in their current state for an extended period of time to maintain the integrity of the cell sample for later observation or testing by a researcher or medical professional.

The sample repository 620 may take any convenient physical form, such as a well formed into the surface of microfluidic device 602, which may remain open or may include a cover that is glued in placed, snapped in place with resilient members that engage the microfluidic device 602, slide in place under guides that extend from the microfluidic device 602 surface, corresponding to the cover variations illustrated in FIGS. 3A-D, or any other convenient means as would occur to one of ordinary skill in the art. The above examples are intended to be only non-limiting examples of many possible configurations.

While the disclosure has been illustrated and described in detail in the drawings and foregoing description, the same is to be considered as illustrative and not restrictive in character, it being understood that only the preferred embodiments have been shown and described and that all changes and modifications that come within the spirit of the disclosure are desired to be protected.

What is claimed:

1. A microfluidic device, comprising:
a substrate;
a microfluidic flow channel formed in said substrate, wherein said flow channel extends through a portion of said substrate adapted to facilitate cytometry analysis of cells flowing in said flow channel; and
a sample repository onboard said substrate and containing material operative to preserve cells in a tissue sample placed within said sample repository.

2. The microfluidic device of claim 1, wherein a location of said sample repository is selected from the group consisting of: on said substrate and in said substrate.

3. The microfluidic device of claim 1, wherein said material is selected from the group consisting of: chemicals and reagents.

4. The microfluidic device of claim 1, wherein said sample repository comprises a well formed in said substrate.

5. The microfluidic device of claim 4, further comprising:
a cover affixed to said substrate and substantially sealing said well.

6. A method for analyzing cells, comprising the steps of:
a) providing a tissue sample;
b) disassociating cells from said tissue sample;
c) analyzing said disassociated cells by cytometry while said cells are onboard a microfluidic device having a substrate; and
d) placing a non-disassociated portion of said tissue sample in a sample repository onboard said microfluidic device.

7. The method of claim 6, wherein a location of said sample repository is selected from the group consisting of: on said substrate and in said substrate.

8. The method of claim 6, further comprising the step of:
e) placing material in said sample repository, said material operative to preserve cells in said tissue sample placed within said sample repository.

9. The method of claim 8, wherein said material is selected from the group consisting of: chemicals and reagents.

10. The method of claim 8, wherein step (e) is performed prior to step (d).

11. The method of claim 6, further comprising the step of:
e) placing a cover over said sample repository.

12. The method of claim 6, further comprising the step of:
e) after step (c), conducting a morphological review of said non-disassociated portion of said tissue sample in said sample repository.

13. The method of claim 6, further comprising the step of:
e) disassociating cells from said non-disassociated portion of said tissue sample in said sample repository; and
f) testing said cells disassociated at step (e).

14. The method of claim 13, wherein step (f) further comprises conducting a cytometry analysis on said cells disassociated at step (e).

15. A microfluidic device, comprising:
a substrate;
a sample well onboard said substrate for holding a tissue sample;
means for disassociating cells from said tissue sample while said tissue sample is in said sample well; and
a microfluidic flow channel formed in said substrate and operatively coupled to said sample well for receiving said disassociated cells, wherein said flow channel extends through a portion of said substrate adapted to facilitate cytometry analysis of said cells flowing in said flow channel.

16. The microfluidic device of claim 15, wherein a location of said sample well is selected from the group consisting of: on said substrate and in said substrate.

17. The microfluidic device of claim 15, wherein said means for disassociating cells comprises:
an input port operatively coupled to said substrate and operatively coupled to said sample well for transfer of fluid thereto;
a supply of chemicals coupled to said input port;
wherein said chemicals are operative to disassociate cells from said tissue sample while said tissue sample is in said sample well.

18. The microfluidic device of claim 15, wherein said means for disassociating cells comprises:
a source of vibratory energy operative to apply at least a portion of said vibratory energy to said tissue sample in said sample well;
wherein said vibratory energy is operative to disassociate cells from said tissue sample while said tissue sample is in said sample well.

19. The microfluidic device of claim 18, wherein said source of vibratory energy produces ultrasonic energy.

20. A method for analyzing cells, comprising the steps of:
a) placing a tissue sample in a sample well onboard a microfluidic device;
b) disassociating cells from said tissue sample within said sample well; and
c) analyzing said disassociated cells by cytometry while said cells are onboard said microfluidic device.

21. The method of claim 20, wherein a location of said sample repository is selected from the group consisting of: on said substrate and in said substrate.

22. The method of claim 20, wherein step (b) comprises applying a chemical to said sample well to disassociate said cells from said tissue sample.

23. The method of claim 20, wherein step (b) comprises applying vibratory energy to said sample well to disassociate said cells from said tissue sample.

24. The method of claim 20, wherein step (b) comprises applying a chemical and vibratory energy to said sample well to disassociate said cells from said tissue sample.

25. A microfluidic device, comprising:
a substrate;
an input port operatively coupled to said substrate for accepting a quantity of cells;
a microfluidic flow channel formed in said substrate, wherein said flow channel extends through a portion of said substrate adapted to facilitate cytometry analysis of said cells flowing in said flow channel; and
a sample repository onboard said substrate and in fluid communication with said microfluidic flow channel;
wherein a portion of said cells may be routed to said sample repository through said flow channel without undergoing cytometry analysis.

26. The microfluidic device of claim 25, wherein a location of said sample repository is selected from the group consisting of: on said substrate and in said substrate.

27. A method for analyzing cells, comprising the steps of:
a) providing a quantity of cells into a microfluidic flow channel formed in a substrate of a microfluidic device;
b) depositing a first portion of said cells in a sample well onboard said substrate and in fluid communication with said microfluidic flow channel; and
c) analyzing a second portion of said cells by cytometry while said cells are onboard a microfluidic device.

28. The method of claim 27, wherein a location of said sample repository is selected from the group consisting of: on said substrate and in said substrate.
